(19) United States
(12) Patent Application Publication
(10) Pub. No.: US 2002/0164777 A1
(43) Pub. Date: Nov. 7, 2002

(54) Devices and methods for high throughput patch clamp assays

(76) Inventors: Andrew J.G. Kelly, Palo Alto, CA (US); Haim Kedar, Palo Alto, CA (US); Larry C. Mattheakis, Cupertino, CA (US); Kerwin C. K. Ng, San Francisco, CA (US); Alexei N. Savtchenko, Palo Alto, CA (US)

Correspondence Address: BROBECK, PHLEGER & HARRISON LLP, 12390 EL CAMINO REAL, SAN DIEGO, CA, 92130, US

(21) Appl. No.: 10/029,425

(22) Filed: Dec. 21, 2001

Related U.S. Application Data
(63) Continuation-in-part of application No. 09/779,955, filed on Feb. 9, 2001.

(51) Int. Cl. C12M 1/18

(52) U.S. Cl. 435/287.1; 435/29; 435/288.4

(57) ABSTRACT

A device for measuring electrophysiological properties of a cell membrane of an individual cell comprises a plate provided with at least one opening. The opening is bounded by a surface and the surface is modified, such as via heat treatment, to facilitate formation of a gigaseal. A chamber is adjacent to the plate. The chamber is in fluid communication with at least one opening and is adapted to hold an electrically conductive solution. The plate further comprises a first electrode located in the chamber and a second electrode located adjacent to the plate.

### RELATED APPLICATIONS

This application is a continuation-in-part (“CIP”) of U.S. patent application Ser. No. 09/779,955, filed Feb. 9, 2001, entitled “Device and Technique for Multiple Channel Patch Clamp Recording,” which is incorporated herein by reference in its entirety.

### FIELD OF THE INVENTION

This invention relates generally to electrophysiological evaluations of biological materials. More specifically, the invention relates to devices and techniques for measuring and evaluating electrophysiological properties associated with ion channels in cell membranes. The invention is further related to techniques for creating a gigaseal between a cell membrane and the surface of a patch clamp probe to facilitate high throughput measurements of electrophysiological properties.

### BACKGROUND

Ion channels control the flow of ions in and out of cells. Typically made of proteins or assemblies of proteins, ion channels are imbedded in lipid bilayers that comprise cell membranes. The movement of ions through cell membranes via ion channels creates ionic currents that give rise to weak but measurable electrical currents.

The patch clamp method enables the measurement of ionic currents flowing through ion channels. A patch clamp technique is described in for example, PCT publication serial nos. WO 96/13721 and WO 99/66329, which are incorporated herein by reference in their entirety. In brief, the patch clamp method uses the ability of a cellular membrane to form a tight seal between the membrane and the recording probe, thus minimizing background ionic currents from “leakage” between the cell membrane and the recording probe. In current patch clamp technology, a micropipette tip engages a membrane and forms a seal. Such a seal, known in the art as a “gigaseal,” has a high resistance that facilitates precise measurement of weak ionic currents flowing through ion channels in the cell membrane.

Many ion channels have “gates” that open in response to external stimuli. External stimuli may include electrical potentials, mechanical or tactile stimuli, and signaling molecules. Signaling molecules are essentially chemical stimuli, and classes of ion channel gates, which respond to chemical stimuli, are known in the art as ligand-gated ion channels. Ligand-gated ion channels respond to both naturally occurring signaling molecules and to synthetic molecular signals such as drugs. Examples of signaling molecules for ligand-gated channels include acetylcholine and glycine (neurotransmitters), cyclic AMP, inositol 1,4,5 triphosphate (IP₃), and ATP (intracellular). The development of effective drugs for the treatment and management of a host of ion-channel related diseases and disorders has been confirmed by patch clamp assays.

In its existing form, the patch clamp method is a low throughput assay for drug candidates. A major bottleneck concerns the formation of a gigaseal between the membrane and the tip of a pipette. Current technology for forming a gigaseal is tedious and requires special training and equipment. An experienced electrophysiologist now can screen only about 5 to 20 compounds a day using existing patch clamp techniques, whereas modem drug screening (e.g., using non-patch clamp techniques, and characterized by 96-well plates, robotic handling, and automated data processing) can screen thousands or tens of thousands (or greater) of compounds per day depending on the particular assay.

Other existing methods of electrophysiological recordings include the use of a two-microelectrode voltage clamp, extracellular recordings, and the “U-tube” method. Although less demanding in terms of equipment and personnel training, these techniques also do not satisfy the current requirements for high throughput screening.

Alternative methods of recording ion channel activity, such as optical methods of recording the voltage change across the cell membrane, have higher throughput. However, these methods lack the precision and the information content of the electrophysiological methods for screening purposes and cannot provide the amount of information one can gain from electrophysiological recordings.

Accordingly, there is a long felt need for a system and method for measuring and evaluating electrophysiological properties of cells and cell membranes under high-throughput conditions, e.g., systems and methods that significantly boost the rate at which patch clamp type assays are performed.

### SUMMARY OF THE INVENTION

According to an embodiment of the invention, devices and methods for enabling automated ion channel assays and the parallel processing and screening of many drug candidates and many cells at once, utilizing a gigaseal between a cell and an opening in a glass sheet or plate, are provided.

An embodiment of the present invention comprises a device for measuring electrophysiological properties of a cell membrane of an individual cell, the device comprising: a plate provided with at least one opening, wherein the opening is bounded by a surface and wherein the surface is modified to facilitate formation of a gigaseal; a chamber adjacent to the plate, wherein the chamber is in fluid communication with at least one opening and is adapted to hold a solution; a first electrode; a second electrode; and wherein electrophysiological properties of a cell membrane of an individual cell is measured using the first electrode and the second electrode.

Another embodiment of the present invention comprises a device for measuring electrophysiological properties of a cell membrane of an individual cell, the device comprising: a plate provided with at least one well, wherein the well is provided with an opening modified to receive an individual cell, wherein the opening is created using a laser and the opening is modified via heating; a chamber adjacent to the plate, wherein the chamber is in fluid communication with the opening and is adapted to hold an electrically conductive solution; a first electrode located in the chamber; a second electrode located in the well; and an amplifier in electrical contact with the first and second electrodes, wherein electrophysiological properties of a cell membrane of the individual cell are recorded by measuring a current through the first and second electrode.

Another embodiment of the present invention comprises a removable disk comprising an opening wherein the disk serves as part of a well for use in measuring electrophysiological properties of a cellular membrane.

Another embodiment of the present invention comprises a method for evaluating currents flowing through ion channels of a cellular membrane, the method comprising: providing at least one well comprising an opening having a modified surface to receive a cell comprising a cellular membrane; depositing the cell onto the opening wherein the modified surface creates a gigaseal between the cell and the well; and recording voltage and/or current measurements to evaluate the ion channel of the cell membrane.

Another embodiment of the present invention comprises a method for creating a gigaseal, the method comprising: providing at least one well comprising an opening; depositing a solution comprising a plurality of cells into the well; providing a positive pressure to the opening; and providing a vacuum to the opening, creating a gigaseal between one of the plurality of cells and the opening.

A technical advantage of one embodiment of the present invention is that a device that facilitates the formation of a gigaseal is provided. Such device provides enhanced signal detection and amplification for the measurement of ionic current. Further, such device provides features that enhance high throughput screening of drug candidates. Additionally, methods of fabricating the device and components of the disclosed invention are disclosed.

A technical advantage of an embodiment of the present invention is that novel ways to create an electrically resistive gigaseal between a cell membrane and the recording probe to facilitate the measurement of ionic currents flowing through a cell membrane are provided. Further, methods of chemically and physically modifying the surface of the probe, which engages the cell membrane, are disclosed. Surface modifications that facilitate the formation of a gigaseal include, but are not limited to: (1) heat treatment for specific time periods, (2) the covalent binding of lipid molecules, and (3) the application of a glue-like substance. In one embodiment, such modifications are used with existing patch clamp type experiments, and may also be used to facilitate high throughput screening procedures.

A technical advantage of an embodiment of the present invention is that novel ways to screen ion channels in a high throughput fashion are provided. Such screening techniques may utilize gigaseals.

Another technical advantage of one embodiment of the present invention is that the number of whole cell patches that may be assayed is increased from the current 5 to 20 per eight hour day to 200 to 2000 (or greater) per day.

Other objects, features, and technical advantages of the present invention will become more apparent from a consideration of the detailed description herein and from the accompanying drawings.

### BRIEF DESCRIPTION OF THE DRAWINGS

Reference is now made to the following description and the accompanying drawings, in which:

FIG. 1 illustrates a device for measuring electrophysiological properties according to an embodiment of the invention;

FIG. 2 is front perspective view of a high throughput screening device according to an embodiment of the invention;

FIG. 3 is a cross sectional schematic diagram of the screening apparatus of FIG. 2;

FIG. 4 is a front perspective view of a multi-well plate that is used in connection with the screening device of FIG. 3;

FIG. 5 illustrates another embodiment of an electrophysiological measuring device according to an embodiment of the invention;

FIG. 6 is a more detailed view of an opening in one of the wells of the screening device of FIG. 5;

FIG. 7 illustrates the screening device of FIG. 6 after a cell has been drawn into the opening according to one embodiment of the invention;

FIG. 8 illustrates the common electrode that is translated to sever a portion of the cell that is sealed to the through opening according to one embodiment of the invention;

FIG. 9 illustrates the common electrode when moved back to its home position so that measurements of electrophysiological properties may be taken according to one embodiment of the invention;

FIG. 10 is a top perspective view of one embodiment of a multi-well plate that may be used in a high throughput screening device according to an embodiment of the invention;

FIG. 11 is a top view of the multi-well plate of FIG. 10;

FIG. 11A is a more detailed view of a well of the multi-well plate of the FIG. 11 taken along detail A;

FIG. 11B is a more detailed view of the well of FIG. 11A taken along detail B;

FIG. 11C is a cross sectional side view of one of the wells of the multi-well plate of FIG. 11;

FIG. 11D is a more detailed view of an opening in the well of FIG. 11C taken along detail D;

FIG. 11E is a more detailed view of the opening of FIG. 11D taken along detail E;

FIG. 11F is another embodiment of an opening in the plate which is substantially less conical in shape than that in FIG. 11E;

FIG. 12 shows a two stage opening comprising a counter bore and a through hole according to an embodiment of the present invention;

FIG. 13A is an oblique view of a multi-well plate according to an embodiment of the present invention;

FIG. 13B is an exploded view of a multi-well plate comprising a glass plate and a plastic sheet also showing a test vacuum fixture according to an embodiment of the present invention;

FIG. 13C shows an oblique close-up view of one well of a multi-well plate according to an embodiment of the present invention;

FIG. 13D shows another oblique close-up view of one well according to an embodiment of the present invention;

FIG. 14 illustrates a patch clamp micro chamber according to one embodiment of the invention;

FIG. 15 illustrates a patch clamp micro chamber with a glass disk according to one embodiment of the invention;

FIG. 16 illustrates the covalent attachment of a lipid to a plate surface according to one embodiment of the invention;

FIG. 17 illustrates the binding of lipid molecules near the opening in a patch clamp device according to one embodiment of the invention;

FIG. 18A illustrates a gigaseal formation according to one embodiment of the invention;

FIG. 18B illustrates a gigaseal formation according to another embodiment of the invention;

FIG. 19A illustrates a patch clamp device with a SQUID detector according to an embodiment of the invention; and

FIG. 19B illustrates a patch clamp device with a SQUID detector according to another embodiment of the invention.

### DESCRIPTION OF THE SPECIFIC EMBODIMENTS

The following detailed description refers to the accompanying drawings. Other embodiments are possible and modification may be made to the embodiments without departing from the spirit and scope of the invention. Therefore, the following detailed description is not meant to limit the invention. Rather the scope of the invention is defined by the appended claims.

For convenience in the ensuing description, the following explanations of terms are adopted. However, these explanations are intended to be exemplary only. They are not intended to limit the terms as they are described or referred to throughout the specification. Rather these explanations are meant to include any additional aspects and/or examples of the terms as described and claimed herein.

The term “biological membrane” used herein is a cellular membrane such as found in a whole cell and also includes artificial membranes such as lipid bilayers and other synthetic polymer membranes.

The term “electrophysiological properties” used herein is a measured electrophysicological property of a cell. These properties are measured using ionic current, voltage, or the magnetic field associated with an ion channel located in a cell membrane. Such measurements may be made in the presence (or absence) of indigenous factors like signaling molecules. Such measurements may also occur in the presence (or absence) of exogenous factors like candidate drug molecules or test compounds under screening conditions. Measurements of electrophysiological properties may also include the measurement of potentials across cell membranes or rates of ion migration through ion channels in the presence of indigenous or exogenous factors. The term electrophysiological property may also comprise the distinction of types of ions that flow through ion channels located in biological membranes.

An “electrically conductive solution” is a solution comprising ions or electrolytes. Ions are charged atoms or molecules that bear a positive or negative charge such as cations and anions. Examples of cations include, but are not limited to: sodium (Na⁺), potassium (K⁺), lithium (Li⁺) and other monovalent cations, calcium (Ca²⁺), magnesium (Mg²⁺) and other divalent cations. Examples of anions include, but are not limited to, chloride (Cl⁻), iodide (I⁻), and other halides.

“Ion channels” are transmembrane proteins or assemblies of proteins that are imbedded in lipid bilayers that comprise cell membranes. Ion channels control the flow of ions in and out of cells. Ion channels may show specificity, e.g., they allow only specific ions to pass through cell membranes. Moreover, various diseases and disorders are closely associated with particular ions and their corresponding channels, for example, K-channels, or Na-channels. The movement of ions through cell membranes via ion channels creates ionic currents that create weak but measurable electrical currents. For this reason, the same cell may display different electrophysiological properties in the presence or absence of different ions, e.g., electrophysiological measurements are sensitive to the type of ion(s) present, in addition to being sensitive to parameters such as presence and concentration of molecular signals. Further, a voltage may be applied to induce the opening of ion channels in a cell membrane.

The term “experimental variables” are factors that are operator controlled, e.g. such factors as temperature, duration of experiment (including duration of a current measurement), method of signal detection, and applied voltage. These factors may be changed from experiment to experiment by the operator and such changes may affect the outcome of a particular experiment. Other experimental variables include the presence of, and concentrations of ions (including buffers), molecular signals, or drug candidates under screening conditions. Another experimental variable comprises the type and number of cells present in an experiment.

“Ionic current” is the flow of ions through ion channels. Ionic currents may also refer to ion migration in electrolytic solutions. When current flows in an electrolytic solution, charge may be carried by the motion of both anions and cations. The solvent in electrolytic solutions is often water, however, non-aqueous electrolytic solutions are known to the skilled artisan, and are within the scope of the present invention.

A “patch clamp micro chamber” is a device for measuring electrophysiological properties of biological membranes wherein a gigaseal is formed in a single integrated device. Additionally, in one embodiment, chip signal amplification and/or processing can also occur in this single integrated device.

A “gigaseal” or a “high resistance seal” is a patch clamp seal having minimal background ionic current from “leakage” due to a poor seal. According to a preferred embodiment, a gigaseal is a high resistance seal of greater than about one giga-ohm (1 GΩ). According to another embodiment, a gigaseal is a high resistance seal of between about one giga-ohm (1 GΩ) to about 100 giga-ohm (100 GΩ). According to another embodiment, a gigaseal is a high resistance seal of about one giga-ohm (1 GΩ) to about 50 giga-ohm (50 GΩ). According to another embodiment, a gigaseal is a high resistance seal of about one giga-ohm (1 GΩ) to about 10 giga-ohm (10 GΩ). In another embodiment, a gigaseal is a high resistance seal of about one giga-ohm (1 GΩ) to about 5 giga-ohm (5 GΩ).

A “proton” is a hydrogen atom stripped of its sole electron. In aqueous solution, protons are associated with water molecules and are properly termed “hydronium ions” or H₃O⁺. Frequent confusion of the terms persists in the open literature, especially in discussions of pH. Unless a distinction is drawn, the words proton and hydronium ion are used interchangeably herein.

The devices and methods disclosed herein are useful for the discovery and evaluation of drugs or other therapeutic agents which are effective against ion-channel related diseases. A “therapeutic agent,” e.g., a drug or prodrug, is any compound or formulation thereof which is effective in helping to prevent or treat a disease or condition. “Effective in helping to prevent or treat a disease or condition” indicates that administration in a clinically appropriate manner results in a beneficial effect for at least a statistically significant fraction of patients, such as a improvement of symptoms, a cure, extension of life, improvement in quality of life, or other effect generally recognized as positive by medical doctors familiar with treating the particular type of disease or condition.

Diseases associated with ion channels and ion channel function include the cardiovascular area, including hypertension and cardiac arrhythmias, pain (local anesthetic), diabetes, epilepsy, anxiety, and the like. Each of these diseases or family of diseases tends to be associated with particular ion channels. In one embodiment, the invention provides devices and methods to facilitate high throughput experiments to identify and screen drug candidates for the treatment of ion-channel associated diseases.

The following detailed description refers to the accompanying drawings. Other embodiments are possible and modifications may be made to the embodiments without departing from the spirit and scope of the invention.

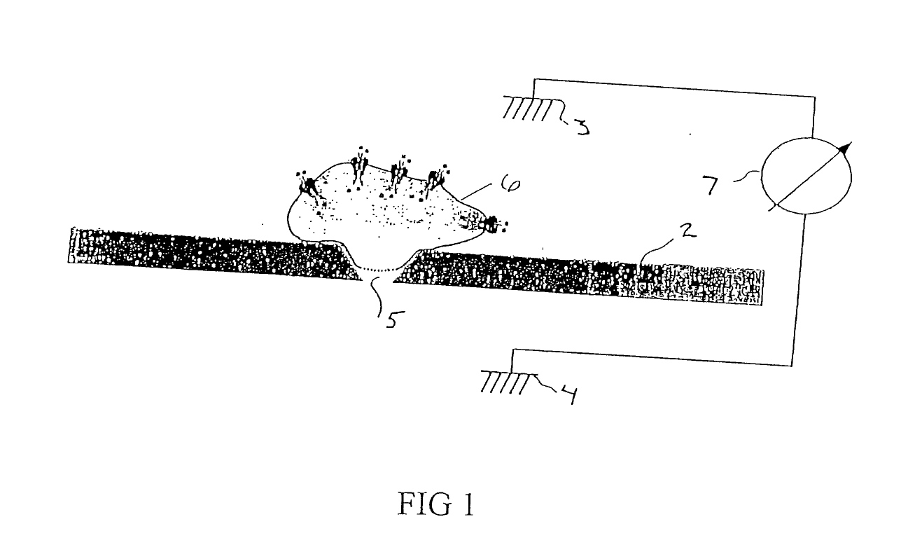

FIG. 1 illustrates a device for measuring electrophysiological properties according to an embodiment of the invention. This device utilizes a dielectric barrier 2 that is used to separate a pair of electrodes 3 and 4. The dielectric barrier may be of any configuration or shape, e.g. a plate, disk or sheet, and may be integrated into another structure, e.g. the bottom, side or top end of a well or a chamber. Hence, a primary function of dielectric 2 is to separate electrodes 3 and 4. Dielectric 2 includes an opening 5 for receiving a cell 6. A variety of methods may be used to shape and form the openings. For example, a small conical hole can be drilled into the bottom of each well using a laser drilling technique. In one embodiment, the opening has a diameter in a range of about 1 μm to about 5 μm, preferably 2 μm.

Once one or more pores are created in cell 6, a measuring device 7 is used to take and record current or voltage values. According to an embodiment of the invention, the dielectric (or multiple dielectrics) includes multiple openings and multiple electrodes so that multiple cells may be evaluated in parallel in multiple wells (one cell at a time).

Dielectric barriers may be constructed from a variety of different materials, for example, glass or plastic. Glass with good insulating electrical properties is useful for patch clamp measurements. Moreover, it is useful to prevent substances from being leached from the glass, like ions, which can alter channel behavior. Other important aspects of the glass are good cell adhesion properties, high electrical resistivity, low dielectric constant, compatibility with laser drilling techniques, uniform thickness, and good flatness.

According to one embodiment of the invention, borosilicate glass is used in the present invention. Borosilicate glass is chemically durable, stable against deformations up to about 700° C., and has excellent optical properties. Thus, this glass is suitable for applications where a sheet of smooth and flat glass with minimal thickness is required. A type of borosilicate glass that can be used is Erie Scientific Company's D 263 glass, which information regarding can be found at: “http://www.eriesci.com/ products&services/CustomGlass/D263-tech.html,” which is incorporated by reference herein in its entirety. Alternatively, other silicate glass or other types of glass may also be used.

According to another embodiment, sodalime glass can be used for the plate portion of a patch clamp micro chamber. For example, microscope slide cover slips can be made from this glass (or borosilicate glass) using a process resulting in flat, uniform, smooth glass sheets.

Alternatively, a silica substrate is used according to an additional embodiment of the invention. Silicon wafers, which are commercially used in the semiconductor industry, have a native oxide layer from air oxidation. Such chips and surfaces offer the advantages of a high quality smooth silica surface that may be functionalized using methods similar to those for the silicate glasses.

Other materials suitable for constructing patch clamp micro chamber components, include, but are not limited to, polymer surfaces. The activation of polymer surfaces may be achieved using a UV/ozone or a plasma process, and involve the creation of new chemical functional groups at the polymer surface, generated through ion and electron bombardment. The process is effective for increasing the surface wettability and also the affinity of charged bipolar lipid membranes of the polymer substrate.

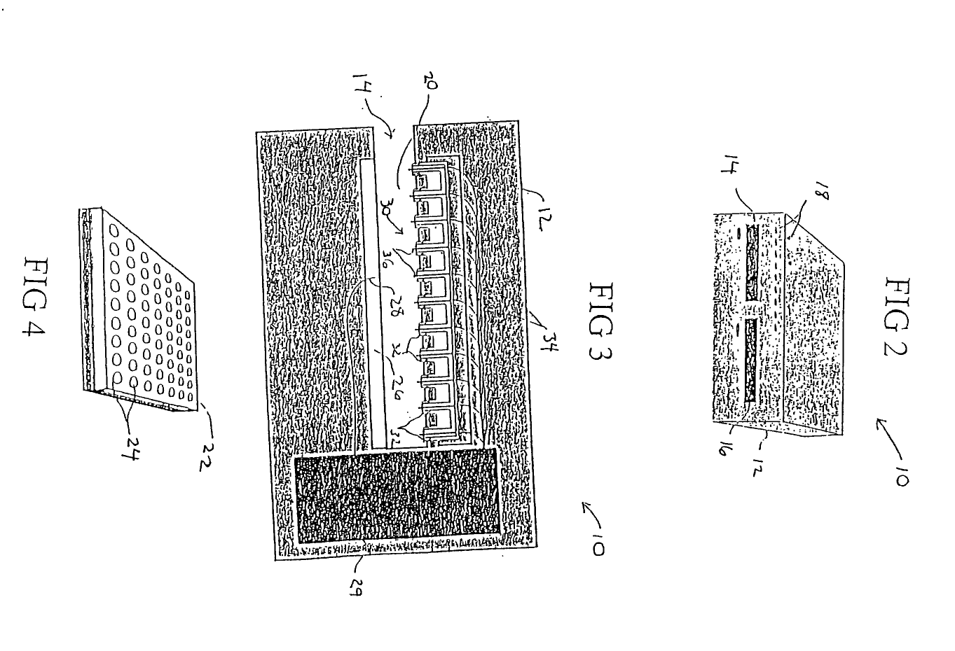

The present invention provides devices and methods for performing multiple channel patch clamp experiments. FIG. 2 shows a front perspective view of such an electrophysiological measuring device 10 according to one embodiment of the invention. Device 10 is constructed of a housing 12 having a pair of inputs 14 and 16 into which multi-well plates (having an array of wells) may be inserted. One type of multi-well plate that may be used according to an embodiment of the present invention is a multi-well plate having a plastic body with a solid glass bottom. An example of such a plate is model No. 7706-2375, commercially available from Whatman Polyfiltronics. One of the plates may include cells while the other plate holds solutions that are transferred to the plate with cells. Positioned above input 14 and 16 are a set of control buttons 18 for controlling operation of device 10. For example, control buttons 18 may be employed to dispense cells into the wells of the multi-well plates, to apply a pressure differential, to create a voltage gradient, to display various measured electrophysiological parameters, and the like. Following evaluation, the multi-well plates may be ejected from housing 12 and discarded.

FIG. 3 is a cross sectional diagram of device 10 according to one embodiment of the invention. Input 14 leads to a generally open interior 20 for holding a multi-well plate 22 having a plurality of wells 24 as in FIG. 4 (a similar interior is in communication with input 16). When plate 22 is positioned within interior 20, it is held over a chamber 26 having a common electrode 28. In use, chamber 26 is filled with an electrolyte solution so that electrical current may be provided through openings in each of wells 24 by energizing common electrode 28 as described herein. Common electrode 28 is coupled to a control unit 29 having the appropriate electronics to provide current to common electrode 28.

A dispensing device is employed to dispense compounds into each well. For example, disposed above interior 20 is a multi-well dispensing device 30 having a plurality of dispensing tips 32. Coupled to each of the dispensing tips 32 is a line 34 leading to a reservoir in control unit 29. Thus, a suspension of cells in solution may be supplied to each dispensing tip 32 which in turn provides the cells (hundreds to thousands of cells) in solution into wells 24 of plate 22.

Electrodes and electronics are provided to measure electrophysiological properties of each cell being studied in each well. For example, each dispensing tip 32 further includes a well electrode 36 that provides a return current path from common electrode 28. In another embodiment, the electrodes are separate from the dispensing tips, may be incorporated into the sides of the wells, may be a thin film electrode on the sides or ends of the wells, or may be placed anywhere that is sufficient to make the measurements herein. Each of well electrodes 36 is coupled to the electronics within control unit 29 so that a voltage gradient may be produced across cell membranes of the cells deposited in each of the openings in wells 24. Further, control unit 29 includes the appropriate electronics to measure and record voltage and current changes for each of the cell membranes.

The device may also include the ability to drain a well or chamber and to maintain a positive pressure or small vacuum to facilitate the formation of a gigaseal. To capture a cell into through openings in each of wells 24, a pressure differential is provided between each well 22 and chamber 26. In one embodiment, this pressure is from 1 to 100 kPa.

Chamber 26 may be pressurized with a slight positive pressure or a slight negative pressure gradient while cells are dispensed into the well to aid in formation of a gigaseal as described below. This may be accomplished by providing positive pressure through each of the dispensing tips 32 or by applying a vacuum within chamber 26. Control unit 29 may control this vacuum.

Control unit 29 further includes appropriate electronics to record and store the electrophysiological properties. Control unit 29 may include appropriate input and output ports to permit this data to be electronically transferred to another computer or other storage device for future use.

Further, control unit 29 may be employed to lower dispensing tips 32 into wells 24 after plate 22 has been inserted into input 14. Following lowering of dispensing tips 32, control unit 29 may then be employed to dispense the cells into solution into each of wells 24 as previously described. Once the operation is complete, control unit 29 can be employed to automatically eject plate 22 from input 14 so that it may be removed and discarded.

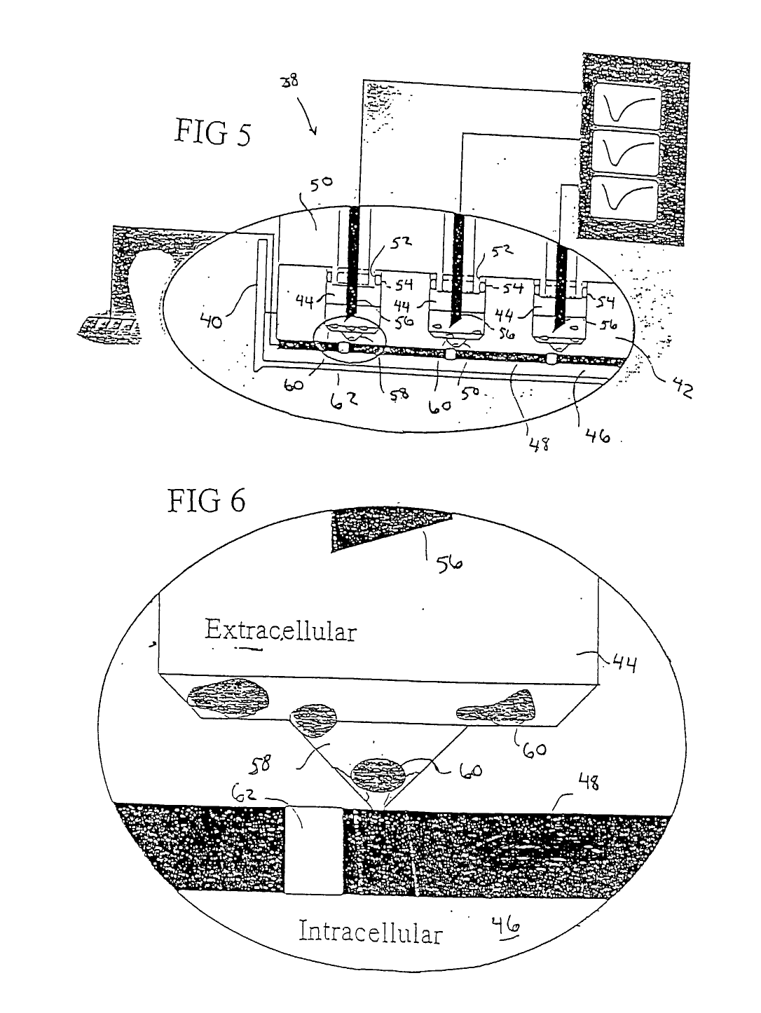

FIG. 5 illustrates another embodiment of an electrophysiological measuring device according to an embodiment of the invention. Device 38 comprises a housing 40 having an interior for holding a multi-well plate 42 having a plurality of wells 44. For convenience of illustration, only three wells are shown. However, it will be appreciated that device 38 can be constructed to have a wide variety of well configurations. In one example, the wells are approximately 800 μm deep and 2 mm in diameter. Further, plate 42 need not be horizontal, but could be positioned at other orientations. Disposed below plate 42 is a chamber 46 for holding an electrolyte solution. Reciprocatably disposed within chamber 46 is a common electrode 48 that is constructed of a metal plate. Electrode 48 is coupled to appropriate electronics to permit a voltage gradient to be applied across cell membranes as described herein.

Disposed above plate 42 is a multi-well dispensing device 50 having a plurality of dispensing tips 52. Dispensing device 50 is configured so that dispensing tips 52 may be inserted into wells 44 after plate 42 is inserted into device 38. Dispensing tips 52 may include a seal 54 to provide a seal between dispensing tips 52 and wells 44 when a pressure differential is applied to wells 44 as described herein. Each dispensing tip 52 further includes a well electrode 56. Thus, a voltage gradient may be provided between common electrode 48 and well electrodes 56 when performing measurements of electrophysiological properties of cells. Electrodes 56 are further coupled to appropriate electronics so that voltage and current measurements may be taken and recorded as illustrated in FIG. 5.

The end of each well 44 includes a tapered through opening 58 to provide a path for electrical current between common electrode 48 and well electrodes 56. With such a configuration, cells 60 may be dispensed into wells 44 using dispensing device 50. Cells 60 are dispensed in a solution that is electrically conductive. Chamber 46 may also be filled with an electrically conductive solution so that a voltage gradient may be applied across the cell membranes of the cells in each well 44.

FIGS. 6-9 sequentially illustrate a method of utilizing device 38 according to embodiments of the present invention. FIG. 6 shows a more detailed view of an embodiment illustrating an opening in one of the wells of the screening device of FIG. 5. Cells 60 in a solution are dispensed into each well 44 using dispensing device 38. Common electrode 48 includes a plurality of openings 62 to correspond with each through opening 58. Initially, common electrode 48 may be shifted so that openings 62 are offset from through opening 58. Thus, the solution in wells 44 will not migrate into chamber 46.

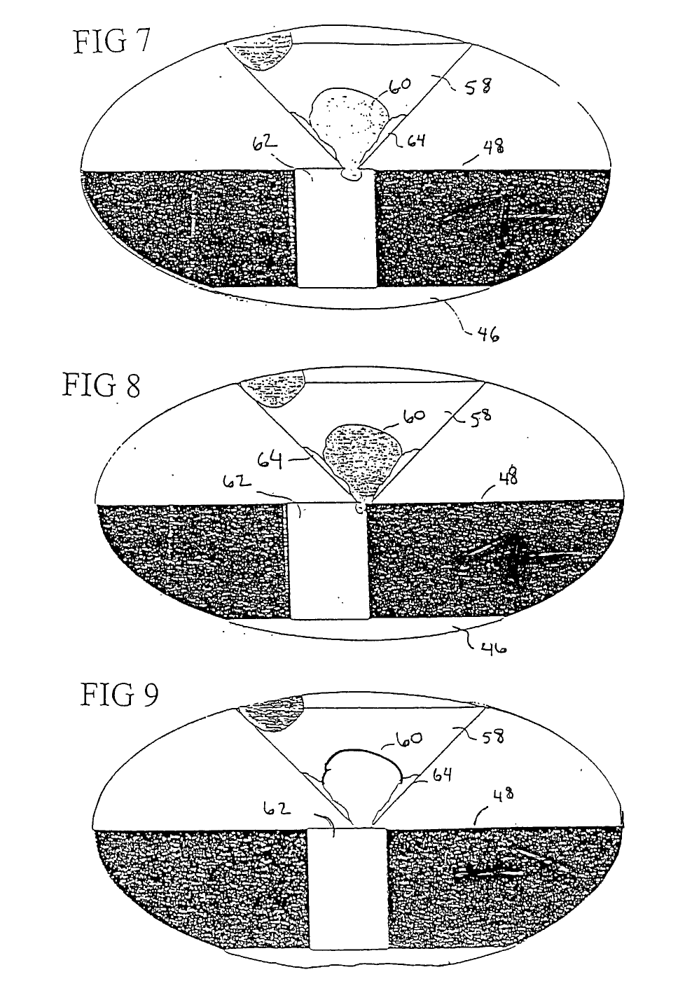

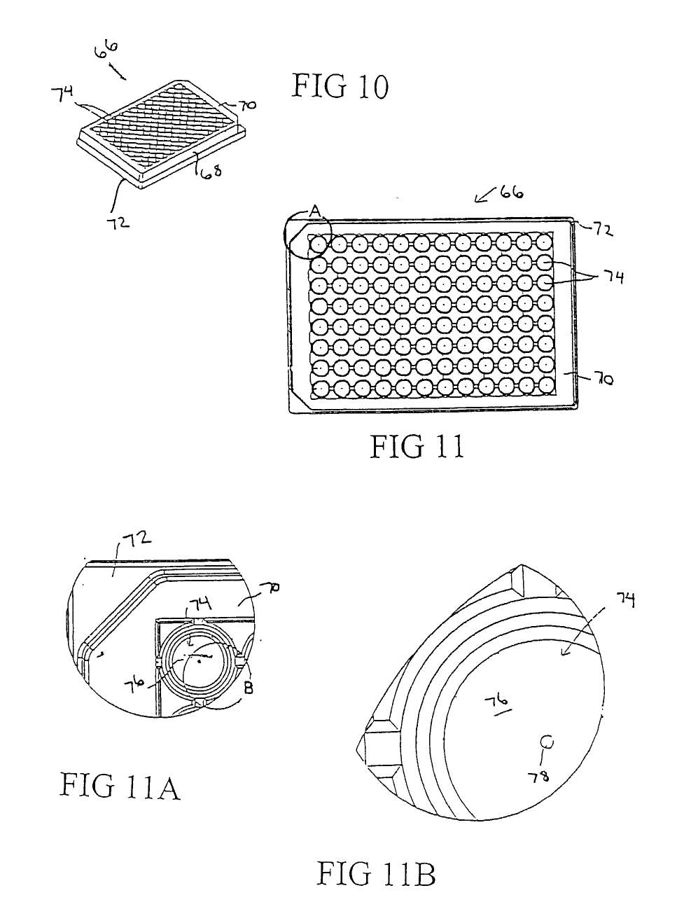

As shown in FIG. 7, electrode 48 is translated to align opening 62 with through opening 58. This causes the solution in wells 44 to flow into chamber 46. Further, a pressure differential may be provided to draw one of the cells 60 to the end of through opening 58 as shown. Such a pressure differential may be provided by supplying positive pressure through dispensing tips 52 and/or by providing a vacuum within chamber 46. The amount of pressure may be varied depending on the type of seal to be created between cell 60 and the side of through opening 58. For example, the side of through opening 58 may optionally include a glue-like substance to create a high resistance seal between cell 60 and the sidewall of through opening 58. Such a glue is illustrated by reference numeral 64 in the figures. A pressure differential may also be provided to provide a gigaseal between cell 60 and the sidewall of through opening 58. Optionally, a potential difference may be provided by applying a voltage difference between the electrodes to determine if an appropriate seal has been created. If not, the wells without a gigaseal are excluded from consideration.

As shown in FIG. 8, electrode 48 may be translated to perforate a bottom portion of cell 60 that extends below through opening 58. Thus, according to one embodiment, the interior part of cell 60 may be placed at the same potential as common electrode 48 when electrode 48 is moved back to the home position and a voltage gradient is applied as illustrated in FIG. 9.

In an optional embodiment of the invention, the cell wall portion that is circumscribed by a high resistance or gigaseal is cut to yield a “penetrated patch.” This may be accomplished, for example, by use of a cutter that is disposed adjacent the plate. The cutter can sever or produce one or more openings in cells protruding below the ends of the wells. A common electrode may be configured to function as the cutter. Thus, the interior of the cell may be placed at the same electrical potential as one of the common electrodes. Alternatively, the bottom of the cell may be perforated using pressure or electrical pulses or by using a Nystatin or other opening forming solution comprising an antibiotic. Further, as an alternative to using electrode 48 as a cutter, device 38 may utilize large pressure pulses to destroy the bottom portion of cell 60 or may use a Nystatin solution to create holes in the bottom portion of cell 60.

In the position shown in FIG. 9, measurements of electrophysiological properties may be made by applying a voltage gradient and measuring the current flowing through the ion channels in the cell membrane. Hence, by utilizing device 38, multiple cells may be evaluated in parallel in a high throughput manner. Once the measurements are made, plate 42 may be removed and discarded.

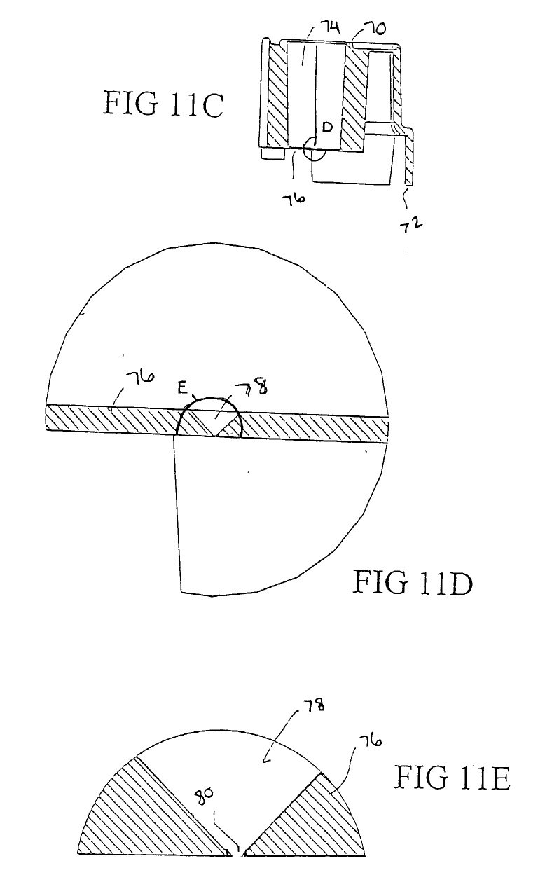

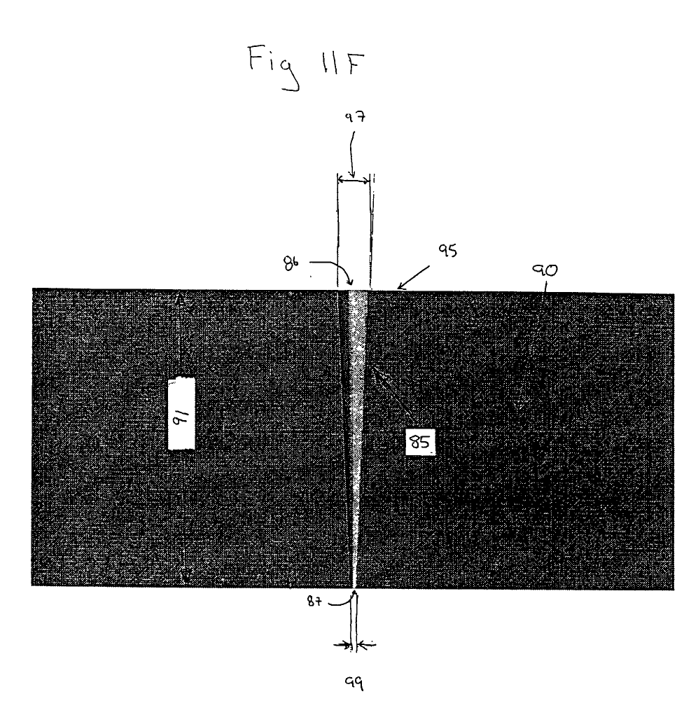

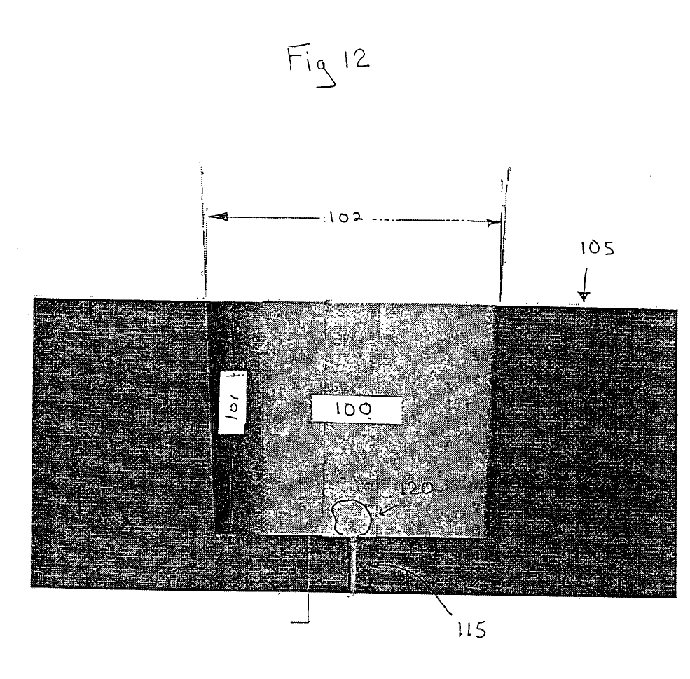

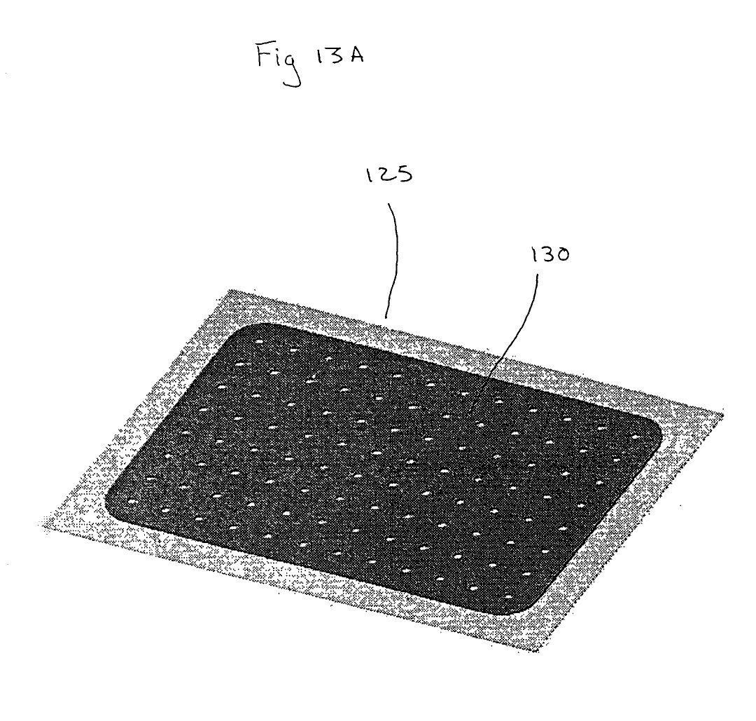

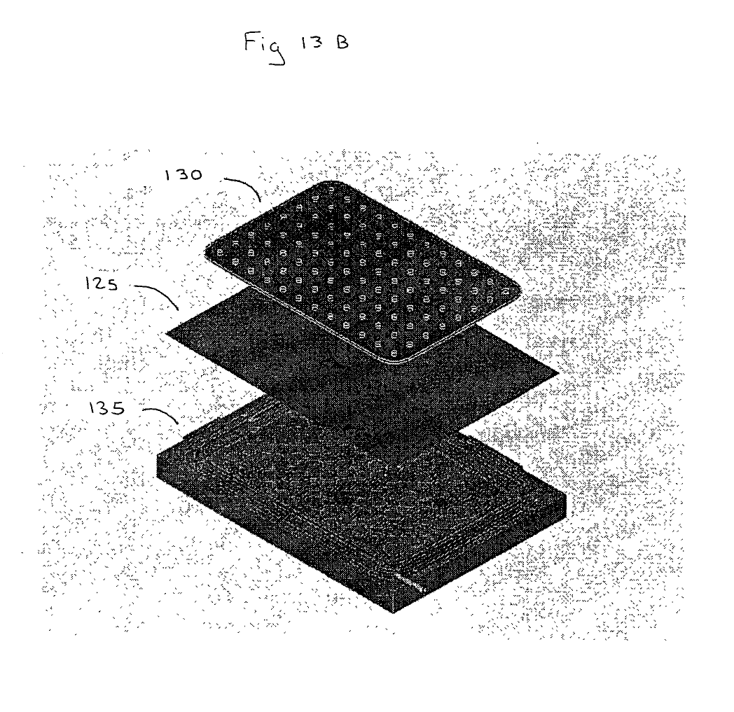

FIG. 10 shows one embodiment of a multi-well plate 66 that may be used with any of the measuring devices of the invention. Plate 66 is constructed of a plate body 68 having a “top” end 70 and a “bottom” end 72. A plurality of wells are formed in the plate body, with each well being open at end 70. Further, and as shown in FIG. 11, each well 74 has a “bottom” end 76. Plate body may be constructed of plastic, with end 76 being constructed of glass. For example, a glass sheet may be bonded to the bottom of polystyrene 96 well plate. Thus, plate 66 is relatively inexpensive to manufacture and may be discarded after use.

FIGS. 11 and 11A-11E show more detail of an embodiment of a multi-well plate 66 that may be used with any of the measuring devices of the invention. FIG. 11A shows a more detailed view of a well 74 of the multi-well plate of FIG. 11 as shown in detail A. FIG. 11B is a more detailed view of the well 74 of FIG. 11A as shown in detail B. FIG. 11C is a cross sectional side view of one of the wells 74 of the multi-well plate of FIG. 11. FIG. 11D is a more detailed view of an opening in the well of FIG. 11C as shown in detail D. FIG. 11E is a more detailed view of the opening of FIG. 11D as shown in detail E.

Several different types of holes with different geometries are used according to embodiments of the present invention. In one embodiment (FIG. 11E), a conical opening 78 having diameter of approximately 30 μm which narrows to a through hole 80 that is approximately 2-5 μm in diameter. Formed in each bottom end 76 is an opening 78 to receive a cell. The taper angle of opening 78 and size of through hole 80 may be varied to optimize the gigaseal according to cell type and average cell size, for example, the tapered angle can be from approximately 1° to 90°. In one embodiment of the invention, the cells have an average diameter of about 8 μm to 80 μm. In another embodiment, the cells have an average diameter of about 8 μm to 12 μm, or alternatively 10 μm to 12 μm.

One technique for forming through opening 78, according to an embodiment, is by using a laser drilling process or related technique. Laser drilling is the process of repeatedly pulsing focused laser energy at a material, vaporizing layer by layer until a through-hole is created. This process creates what is known as a “popped” or “percussion drilled” hole. Depending upon material and material thickness, a popped hole could be as small as 1 μm in diameter. If a larger hole is required (such as larger than 100 μm in diameter), the laser, once through the material, can be moved with respect to the work piece to contour the desired diameter. The end result is a fast, efficient way to create quality holes. Preferably, an ultraviolet laser is used to create these holes, however, an infrared laser can also be used. Additionally, the laser drilling process can be automated to more ensure accurate and precise drilling of the holes.

FIG. 11F is another embodiment of an opening in a plate. In FIG. 11F, the opening 85 in the plate 90 is substantially less conical in shape, deviating by only a several degrees from a line drawn perpendicular to the surface. An opening 85 may be further characterized as having a “large” end 86 and a “small” end 87, each having different diameters 97 and 99, respectively. According to one embodiment of the invention, opening 85 has a diameter of approximately 7 to 9 μm on larger side 86 and approximately 1-3 μm on the small side 87. In this embodiment, the thickness 91 of the glass plate in FIG. 11F is approximately 100 μm.

In one embodiment of the invention, the larger end of conical-shaped opening engages the cell and the smaller end opens to the chamber as illustrated in FIG. 1. In another embodiment of the invention, the large and small ends are reversed and such that the smaller end of the opening engages a cell.

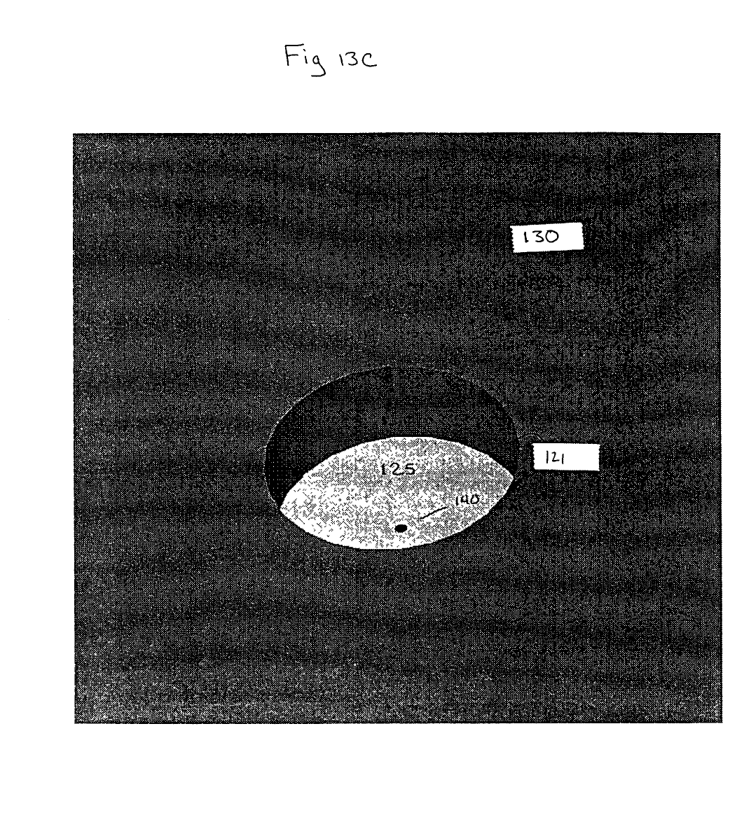

FIG. 12 shows another embodiment of the invention, wherein a hole is constructed in two stages with a counter bore and a through hole. A relatively large (with a diameter 102 of approximately 80-100 μm) opening, the counter bore (or blind hole) 100 is drilled using a mask partway through a 100 to 120 μm thick glass disk 105 to a depth 101 of approximately 80 to 100 μm in 100 μm thick glass. Alternatively, the counter bore 100 is drilled without use of a mask. A second slightly conical shaped “through hole” 115 is then drilled through approximately 15-20 μm of glass that remains at the bottom of the counter-bored hole, having a diameter of approximately 2 μm. The laser drilled holes in this embodiment are slightly tapered, having an angle of 2 to 5 degrees. This slight tapering is caused by the laser drilling process. A cell 120 is significantly smaller than the counter bore yet larger than the opening of the through hole and seats atop the through hole when forming a gigaseal.

As stated above, in one embodiment, the counter bore 100 and the through hole 115 are drilled using a laser. The laser has a wavelength of 193 nm for the counter bore 100 and 248 nm the through hole 115. Alternatively, the laser has a wavelength between approximately 150 and 300 nm for both the counter bore 100 and the through hole 115. The wavelength that is used to drill the hole is determined by the type of glass (or other material) that is being used. Some glass does not absorb well at 248 nm, so a 193 nm laser may be used. In another embodiment, both the counter bore 100 and through hole 115 can be drilled using the same wavelength laser, such as a 248 nm laser. If using the same laser, different masks may be used to change the laser beam diameter.

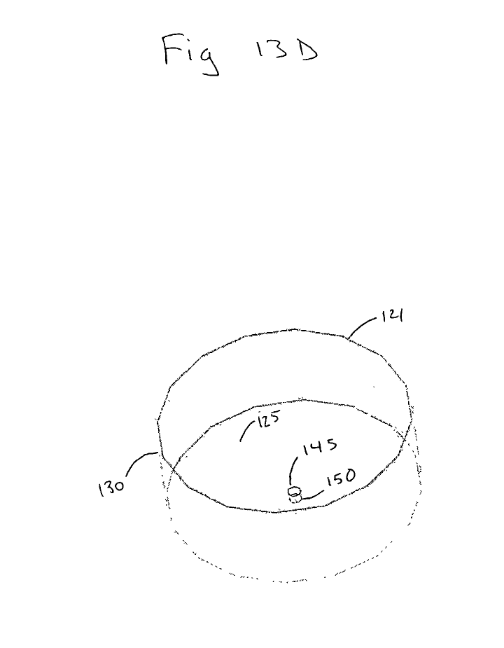

FIG. 13A shows an oblique view of another embodiment of a multi-well plate, such as a composite 96-well plate, according to an embodiment of the invention. A glass plate or sheet 125 is provided with a plurality of openings. The openings may be made using any of the methods described herein. A layer of plastic 130, e.g. a silicone elastomer, which is also provided with an array of larger holes, may be overlaid or adhered to the glass plate. The resulting glass/plastic composite comprises a multi-well plate according to an embodiment of the invention.

FIG. 13B is an exploded view of a multi-well plate comprising a glass plate and a plastic sheet also showing a test vacuum fixture according to an embodiment of the present invention. FIG. 13B also shows the lower chamber which further comprises vacuum fixture 135.

FIG. 13C shows an oblique close-up view of one well 121 of a multi-well plate according to an embodiment of the invention. The well 121 comprises a plastic layer 130 adhered to a glass plate or sheet 125, which is provided with an opening 140. Opening 140 can comprise a counter bore and a through hole. The sides of well 121 therefore comprise plastic 130 and the bottom of well 121 comprises glass plate or sheet 125.

FIG. 13D shows another oblique close-up view of one well 121 according to an embodiment of the invention. This “hidden lines visible” drawing of well 121 more clearly shows through hole having top 145 and bottom 150 openings. Again, the sides of well 121 comprise plastic 130 and the bottom of well 121 comprises glass plate or sheet 125.

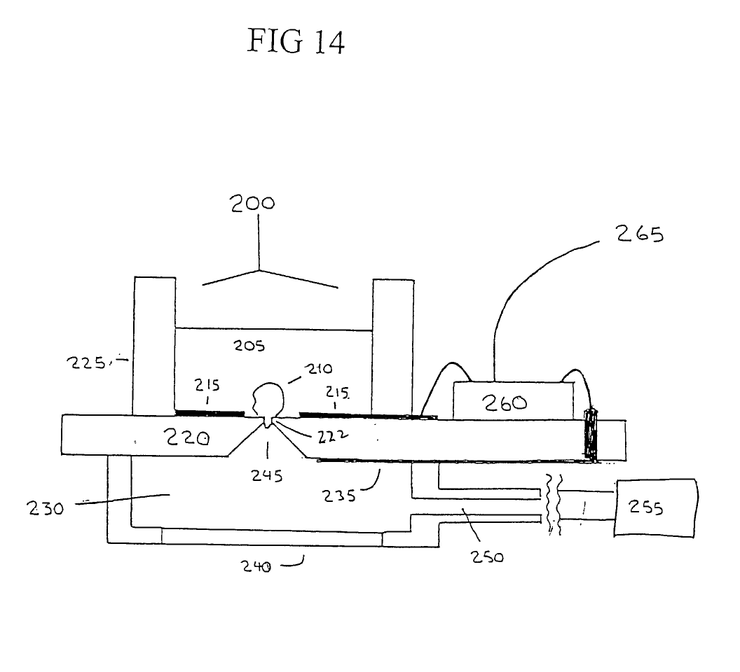

FIG. 14 shows one embodiment of a patch clamp micro chamber according to an embodiment of the invention. The device comprises a well 200 for receiving a solution 205 containing cell(s) 210. According to an embodiment, a well further comprises an electrode 215 that comprises silver coated with silver chloride. In FIG. 14, the electrode appears to rest on plate 220, however, and as described herein, the electrode may be connected to the sidewall of well 225, or in another embodiment, may be part of a liquid dispensing system as shown in FIG. 3. The patch clamp micro chamber according to FIG. 14 further comprises chamber 230 that holds an electrically conductive buffer solution along with a second electrode 235. According to one embodiment of the invention the bottom of the chamber has a window 240 for inspection of the opening 245. The opening 245 receives an individual cell 210, such that measurements are taken on the individual cell 210 as described herein. Such measurements may measure electrophysiological properties of the cell and are recorded using commercially available patch clamp recording electronics designed for pipette-based ion channel recordings, such as a system manufactured by Axon Instruments or HEKA Elektronik.

Referring again to FIG. 14, a patch clamp micro chamber may further comprise a tube 250 leading out of the micro chamber for controlling the pressure of the buffer solution inside the chamber. In one embodiment, a vacuum source 255 is coupled to the micro chamber via a tube 250 to produce a vacuum within the chamber, for example −1 to −15 kPa, alternatively −1 to −5 kPa or alternatively still −1 to −2 kPa. The vacuum (or negative pressure gradient) allows a cell to be sucked to the opening 245, assisting in the formation of the gigaseal.

In another embodiment, a positive pressure is applied through the same tube. Such pressure differentials facilitates the deposition of a cell within the opening and the creation of a gigaseal between a cell membrane and the surface surrounding an opening 222 by blowing clean buffer up through the opening and keeping debris out of the opening until the cell is near the opening. Alternatively, positive pressure may be provided into the well through the opening while cells are being loaded. Also evident in FIG. 14 are the relatively short distances between the cell electrodes 215 and 235 and an amplifier 260 that amplifies the input signal to give a strong signal output 265.

The patch clamp micro chamber shown in FIG. 14 comprises a well for receiving a cell or cells. In one embodiment, the volume of a well in a patch clamp micro chamber device is minimized, being just enough for handling of the test compound and for cell survival. For example, the volume of the well is approximately 300 μL and the bottom of the well has an opening of a few microns diameter.

A patch clamp micro chamber may further comprise a liquid dispensing system that has a dispenser configured to place a cell in solution into a well. Thus, a well may rapidly be provided with a cell using, for example, automated robotics. A first electrode is provided that may be positioned in a well. A well electrode may be coupled to one or more dispensers, such that the placement and removal of an electrode is under control of automated robotics. Each dispenser may include a seal member to form a seal with the well such that positive pressure may be supplied to each well. Alternatively, the liquid dispensing system can dispense liquid to an array of addressable wells, wherein each of the wells is independently addressable by the automated liquid dispensing system. The wells are addressable such that the dispensing system can identify an individual well and place specific cell(s) (or cell type(s)) in a certain well.

In another embodiment, a patch clamp micro chamber comprises a plate having a plurality of wells for receiving cell(s). A patch clamp micro chamber having a plurality of wells may further comprise a common chamber disposed adjacent each opening that is used to hold an electrically conductive buffer solution. In one embodiment, a common electrode is disposed in the chamber, and a plurality of well electrodes are provided that may be positioned within the wells to create a voltage gradient across cell membranes of the cells that are positioned within the openings. Thus, electrophysiological properties of multiple cells may be measured at the same time. In another embodiment, each individual well has a separate chamber.

According to another embodiment of the invention, a multi-channel liquid dispensing system is provided that has a plurality of dispensers that may be configured to place cells in solution into each of the wells. Thus, each well may be rapidly provided with a cell using, for example, automated robotics. According to one embodiment of the invention, the well electrodes may be coupled to the dispensers, such that placement and removal of an electrode is under control of automated robotics. Each dispenser may include a seal member to form a seal with a well such that a positive pressure may be supplied to each well.

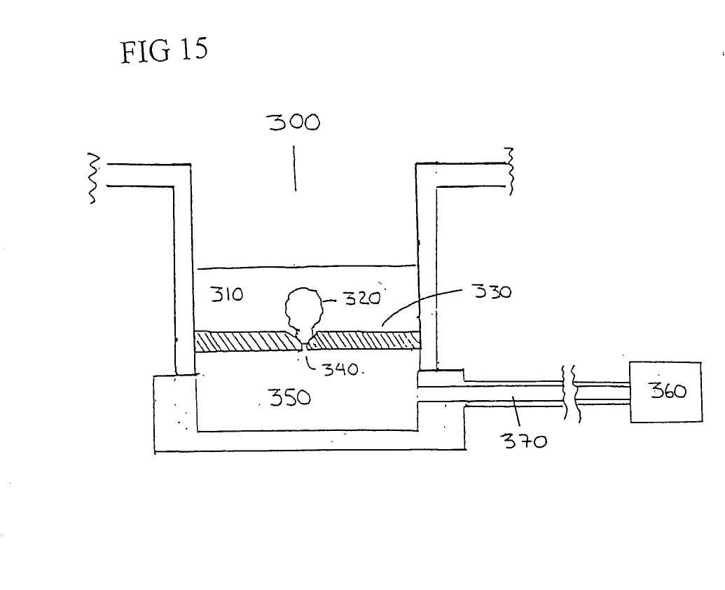

FIG. 15 shows one embodiment of a patch clamp micro chamber with a glass disk according to an embodiment of the invention. The device comprises a well 300 for receiving a solution 310 containing cell(s) 320. The well further comprises a glass disk 330 that is provided with an opening 340 that separates a well from a chamber 350. Electrodes may be configured in a number of ways, for example as shown in FIG. 14. Glass disk 330 fits into the micro chamber and is equipped with one or more openings 340. In one embodiment of the invention, the glass disk 330 is removable from the micro chamber and may be used in another micro chamber or array thereof. In one embodiment, a vacuum source 360 is coupled to the micro chamber via a tube 370 to produce a vacuum within the chamber. Such a vacuum facilitates the deposition of a cell within the opening 340 and creates a high resistance seal.

The surface surrounding an opening in a laser-drilled glass plate or sheet may be modified to enhance the formation of a gigaseal between a cell membrane and the surface of an opening in a well. Such modifications include, but are not limited to, heating (such as via oven baking) the glass plate, adhering a glue-like substance to the surface of the plate surrounding the opening 222, or covalently bonding lipids to the surface of the plate surrounding the opening 222. Alternatively, the surface of the plate can be modified, as described herein, prior to drilling the openings in the well.

In one embodiment, the glass, glass disk, sheet or plate is heat treated before an experiment, such as by heating (for example via oven baking) the glass to near or at the softening temperature of the glass. The softening temperature of a glass is the temperature at which a glass loses enough viscosity that it stops acting like a brittle solid and begins to flow like a liquid. For example, borosilicate glass has a softening temperature of 736° C., so such heat treatment can be at 700° C. for 3 to 10 minutes.

Alternatively, the glass, glass disk, sheet or plate is heated to a temperature sufficient to enhance the formation of a gigaseal between a cell membrane and the surface of an opening in a well, which in one embodiment could be 400° C. The heating temperature and time may further vary depending on the hole geometry, dimensions (for example the depth of counter bore, the conical angle of the through hole, and the like), the type of glass and the thickness of the glass. Because the glass is heated from an external source, and also because the glass is thinnest around the opening, the heat treatment has the effect of modifying the surface surrounding the opening. Heating the glass improves the quality of the gigaseal. In one example, a working patch clamp micro chamber comprising a laser-drilled counter bore and through hole as shown in FIG. 12 had a measured resistance of approximately 725 MΩ before heating. After heating, resistance values can be as high as 10 GΩ. After heat-treating or baking, the glass is ready to be assembled into the micro chamber for a patch clamp experiment.

The softening temperature of the glass can, for example, be determined using the following method, which can be found at “http://enterprise.astm.org/PAGES/C338.htm,” which is incorporated by reference herein in its entirety:

1. This test method covers the determination of the softening point of a glass by determining the temperature at which a round fiber of the glass, nominally 0.65 mm in diameter and 235 mm long with specified tolerances, elongates under its own weight at a rate of 1 mm/min when the upper 100 mm of its length is heated in a specified furnace at the rate of 5+1° C./min.

2. This standard does not purport to address all of the safety problems, if any, associated with its use. It is the responsibility of the user of this standard to establish appropriate safety and health practices and determine the applicability of regulatory limitations prior to use.

In one embodiment of the present invention, the surface of the plate surrounding an opening is modified by the application of a glue-like substance onto a side wall or surface surrounding the opening. This may be accomplished, for example, by dipping the bottom of the multi-well plate in a reservoir containing the glue-like substance and then removing the excess glue by shaking the plate or by applying a small pressure to one side of the plate. Similarly, other known techniques for applying the glue-like substance may be used. Once dispensed in the well, the cells will form a tight sealed contact (a gigaseal) with the wall of each well allowing measurements of electrophysiological properties. The glue-like substance may comprise a silicone-based glue, a Vaseline/paraffin-based composition, or the like. Such a glue-like substance is preferably a chemically inert, soft grease-like substance. This allows the cell to stick to the surface of the through opening and form the gigaseal.

In another embodiment of the present invention, a glue-like substance or lipid surface coating may be placed onto the side wall or surface surrounding the opening. Thus, multiple cells may be simultaneously screened by placing them into individual wells where the high resistance seal is produced between each cell and opening formation of each well. The formation of a plurality of seals also facilitates high throughput screens by enabling multiple seals to be created when simultaneously evaluating multiple cells using electrophysiological techniques.

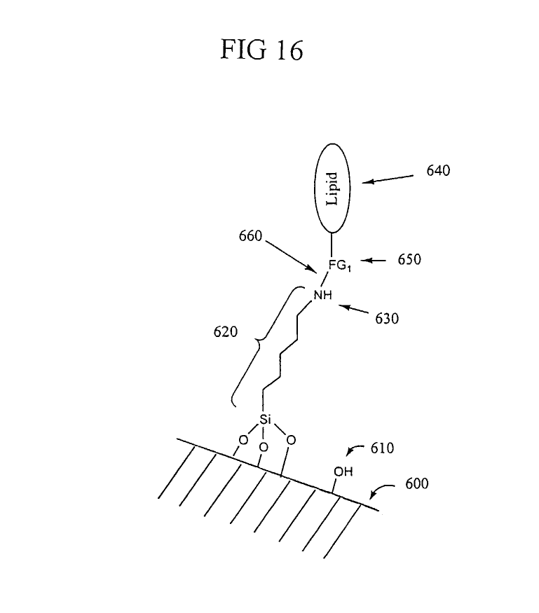

FIG. 16 shows the covalent attachment of a lipid to a plate surface, such as glass, according to one embodiment of the invention. A cell membrane comprises a lipid bilayer. Both layers of the lipid bilayer are made of phospholipid molecules, each having a polar “head” and non-polar “tail.” In water, phospholipid molecules align with their polar heads facing the surrounding aqueous milieu, and the polar end of the second layer exposed to the aqueous milieu of the cytosol. The non-polar ends of each layer are held in contact and comprise the “interior” of the cell membrane.

Bonding a layer of phospholipid to a surface may be accomplished via covalent or non-covalent bonding. For example, a glass surface can be functionalized, e.g., made reactive by attaching an amino-silane moiety to silica and glass surfaces using methods known in the art.

Lipids having a variety of functional groups suitable for covalent coupling to a modified surface are commercially available, for example, from Avanti Polar Lipids, Inc. In one aspect of the invention, such functionalized lipids may be used for binding to modified surfaces of the instant invention. Methods of covalently attaching functionalized molecules to immobilized molecules on for example surface are known to the skilled artisan.

In an embodiment of the present invention, suitable complimentary functional groups on a lipid suitable comprise nucleophiles and carbon electrophiles. The terms “nucleophile” and “electrophile” have their usual meanings familiar to synthetic and/or physical organic chemistry. Carbon electrophiles comprise one or more alkyl, alkenyl, alkynyl or aromatic (sp³, sp², or sp-hybridized) carbon atom substituted with any atom or group having a Pauling electronegativity greater than that of carbon itself. Examples of preferred carbon electrophiles include but are not limited to carbonyls (especially aldehydes and ketones), oximes, hydrazones, epoxides, aziridines, alkyl-, alkenyl-, and aryl halides, acyls, sulfonates (aryl, alkyl and the like). Other examples of carbon electrophiles include unsaturated carbons electronically conjugated with electron-withdrawing groups, examples being the β-carbon in α,β-unsaturated ketones or carbon atoms in fluorine substituted aryl groups. In general, carbon electrophiles are susceptible to attack by complementary nucleophiles, including carbon nucleophiles, wherein an attacking nucleophile brings an electron pair to the carbon electrophile in order to form a new covalent bond between the nucleophile and the carbon electrophile.

According to one embodiment of the invention, suitable carbon electrophiles comprise carbonyls, epoxides, aziridines, cyclic sulfates and sulfamidates, and alkyl, vinyl and aryl halides. According to one embodiment of the invention, suitable nucleophiles comprise primary and secondary amines, thiols, thiolates, and thioethers, alcohols, alkoxides. These nucleophiles, when used in conjunction with preferred carbon electrophiles, typically generate heteroatom linkages (C-X-C) between the homing peptides and scaffold, wherein X is a hetereoatom, e.g, oxygen or nitrogen.

According to another embodiment of the invention, a phospholipid may comprise a photolabile functional group suitable for coupling to a glass plate or other substrate when activated by light.

Phospholipids with either amine or activated carboxyl functional groups, e.g., N-hydroxysuccinyl (NHS) esters, may be coupled to a surface bearing a complementary function group. For example, an amine-bearing lipid will react with a surface bearing NHS ester groups with the concomitant formation of an amide linkage. Likewise, a lipid bearing an NHS ester will react with amine-bearing lipid, also with the concomitant formation of an amide linkage. The choice of which permutation of complementary functional groups depends on the experimental conditions faced, e.g. cell type, other components present, or the like. One advantageous aspect of the present invention is the modular approach to coupling partners. Other complimentary carbon electrophiles and nucleophiles, which may couple to form covalent bonds, may be envisioned by the skilled artisan and fall within the scope of the present invention. For example, phospholipids bearing a thiol (—SH) functional groups may be coupled to a surface also bearing thiols (—SH) via the formation of disulfide bonds.

Referring again to FIG. 16, glass surface 600 comprises hydroxyl groups 610, that may be functionalized with aminosilane 620 using known methods. Other methods known in the art can immobilize other reactive groups on a glass surface. Other functionalized glass surfaces suitable for coupling to functionalized lipids comprise aldehydes, epoxides, maleimides, nickel chelates, streptavidin, biotin, and thiols.

Although the aminosilane shown in FIG. 16 has a hydrocarbon linker having five —CH₂— groups, the skilled artisan will appreciate that the actual linker length is variable. In FIG. 16, the aminosilane terminates in an amino group 630 that is suitable for covalent coupling to a lipid molecule 640 bearing a complimentary electrophillic functional group 650. Subsequent coupling leads to a new covalent bond 660. This surface chemistry produces a high density of lipid molecules on a glass surface.

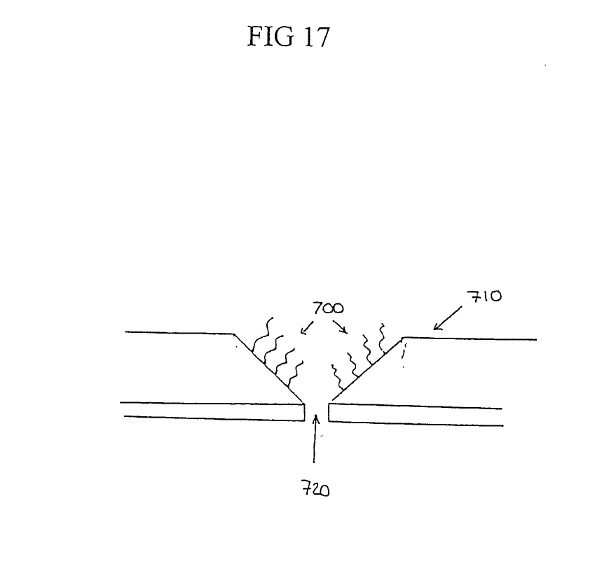

FIG. 17 illustrates the covalent attachment of lipid molecules 700 on a surface 710 surrounding an opening 720 in a patch clamp device according to one embodiment of the invention. In this embodiment, one end of each lipid molecule is attached to the surface, leaving the other end free to interact with a cell membrane. According to one embodiment of the invention, such a covalently bonded layer may dissolve into a membrane. A gigaseal is thus established between the surface and the cell.

Phospholipids may be selectively attached to the surface surrounding the hole. In one embodiment of the invention, holes may be laser drilled in a plate or substrate before covalently attaching a lipid. A lipid may then be to selectively linked using a photo-labile functional group. Other areas of the plate or disk may be photo-masked.

Liquid chemical treatments may also be used to increase the affinity of a cell membrane for a polymer surface. One example is exposure to caustic soda solution. The alkaline solution hydrolyses ester groups at the polymer surface, increasing the wettability and also the surface affinity for charged bipolar lipid membranes.

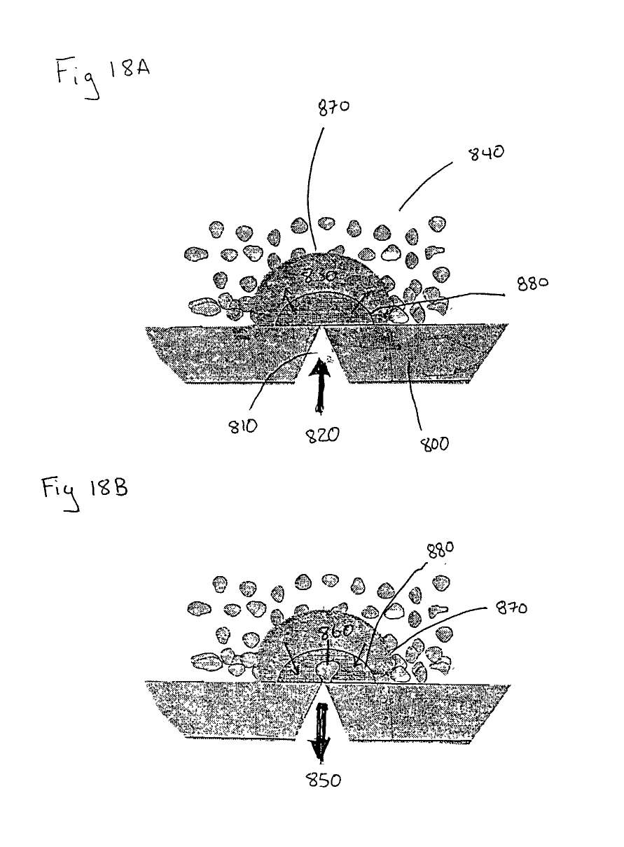

The various embodiments of patch clamp devices disclosed herein comprise pressure control systems. FIGS. 18A and B illustrates gigaseal formation according to an embodiment of the present invention, for example using a pressure control system. A glass plate 800 provided with an opening 810. The opening geometry may be of any type disclosed herein. A positive pressure 820 is applied to the bottom chamber during the filling the upper chamber with a solution comprising cell(s) 840. Without being bound to any particular theory, such conditions have the effect of creating an expanding zone 870 of clean intra cellular solution radiating outward 830 (as shown moving from zone 880 to zone 870) from the hole into the well containing the cell(s). Given micro fluidic properties, the expanding zone of clean fluid is formed without turbulence. Experiments showed that clean fluid can flow through the hole for an extended amount of time without affecting the subsequent gigaseal formation when a cell engages the opening.

Normal healthy cells have a higher protein content than dead or diseased cells, and thus are more dense than debris and dead cells. Because healthy cells are denser, they sink faster than dead or diseased cells. Clumps of cells, which may be just as dense as healthy cells, have a greater hydrodynamic drag to weight ratio than healthy or “good” single cells and also tend to sink more slowly. Due to the expanding zone of clean fluid 830, the debris and clumps are carried farther away than the “good” cells than the debris and the clumps. Therefore, the expanding zone of clean fluid can carry the debris and clumps farther away from the hole than the “good” cells as the collection of cells fall to the bottom.

After the proper configuration is established (FIG. 18B), suction or a negative pressure gradient 850 draws the clean fluid from the zone back through the hole. As the zone shrinks from zone 870 to zone 880, a “good” cell 860 is sucked into the hole before the debris and the clumps, and a gigaseal is established. Thus the gigaseal is formed reliably and a “good” cell remains within zone 880, while the dead or diseased cells and debris remain outside of zone 880.

The following stepwise protocol is an example of a protocol that may be used to reliably form a gigaseal using any of the patch clamp devices disclosed herein. First, the patch clamp chamber is loaded and the electrodes are properly assembled. Second, about 100 μL of cells are dispensed at 5 millions/mL into the top well, which was previously loaded with about 10 μL of Ringer solution (the solution inside of the pipette). Third, a pressure is applied to the bottom chamber. In one embodiment, pressure is applied using a 10 mL syringe. The syringe is compressed to 5 mL from 10 mL for a pressure of about 14 psi. Then the plunger is released. The plunger generally returns to 9.8 mL slowly which is about 0.3 psi. Fourth, wait for 1 minute. One minute is a good example of the amount of time to wait because previous experiments have shown that cells generally settle in 1 minute. Fifth, apply suction at a pressure of −5 kPa.

The above procedure and parameters may be optimized for particular cells and other experimental variables. In another embodiment, the devices and techniques of the invention facilitate the formation gigaseals between a cell and an opening in the wells of a multi-well plate.

The invention further provides methods for automating the screening of ion channel assays and enables the parallel processing of many compounds and many cells at once. The invention may utilize native cell lines for example, CHO, Jurkat, HEK and hERG. Thus, the invention provides the ability to screen the same compound against multiple targets in the same experiment which increases throughput.

Multiple channels permit the screening of the same drug molecule against multiple target ion channels within the same experiment. When used as a drug discovery tool, the invention may be used to determine whether drugs are good modulators of ion channels. The invention allows maximum flexibility and control over the components of each well. The device allows the contents of each well, cell type, drug candidate, buffer and ion concentration etc., to be different depending on the experiment at hand.

The present invention provides the ability to use the ion channels as “biosensors.” In addition to directly affecting gated-ion channels, drugs may affect other molecular targets within a cell and may influence ion channel activity. Effects observed by measurements of electrophysiological properties using devices and methods disclosed herein may be correlated with mechanism of drug action and may be quantified in terms of drug concentrations, e.g., the effectiveness of a particular drug. Such precise measurements are useful when comparing or distinguishing drug candidates to one another.

In one embodiment of the invention, drug-induced modulation of protein kinases and phosphatases within cells, which in turn changes the kinetic behavior of certain ion channels, may be detected and recorded with high precision electrophysiological assays, using the device and methods disclosed herein. Further, some cell lines may be used to evaluate the effect of the same drug on specific kinases, phosphatases, the phosphorylation of ion channels, and an effect of a drug on the channel itself.

In another embodiment, the device may be used to measure precise pH changes. Proton-gated ion channels are selective to protons only, H⁺ or alternatively, hydronium ions H₃⁺O. Such proton selective ion channels are natural pH meters because when two solutions with different ion concentrations are separated by a selective membrane, such as a ion channel, it is possible to observe a concentration as a potential difference. Moreover, when such differences exist, ions will migrate to alleviate the imbalance. This flow may be observed as an ionic current.

Certain commercial pH meters lack precision because the semi-permeable glass membranes which they employ suffer interference by other ions, for example, by sodium Na⁺ ions. Because proton ion channels are selective for protons, an ion channel-based pH meter represents an improvement over the existing state of the art. Thus, the present invention may serve as an alternative to a micro physiometer.

In another embodiment of the present invention, the device and methods disclosed herein may be used to construct a “proton biosensor” for drug discovery. Introduction of certain drugs into a cell may lead to a change in intracellular pH. In still another embodiment, the device may act as a pH meter itself, for example, when the drug is unable to pass through the cell membrane. In this case, measured changes in pH correspond to solution pH within a well.

The electrophysiological information output from a single experiment of the invention can comprise multiple parameters that are recorded essentially simultaneously. The techniques of the invention also provide the ability to dialyze the cell cytoplasm, thus allowing one to manipulate the intracellular solution composition, introducing or removing certain ions from the intracellular solution. In this way, an experiment may dialyze a cell cytoplasm to evaluate one type of channel while excluding other channel types. This permits the optimization of one channel while excluding others. Further, the electrophysiological methods have high sensitivity, allowing one to record the activity of a single channel molecule. The techniques of the invention also have high temporal resolution (in sub-millisecond range) which is useful for some ion channel targets, such as fast deactivating Na channels.

Ionic currents flowing through ion channels are on the order of several pico amperes (pA) and thus are challenging to measure precisely. In one embodiment of the present invention, relatively short distances, e.g., a few millimeters, between the cell electrodes and the amplifier, minimize electrical noise pickup. In one embodiment of the invention, there is just enough distance to connect the electrode inside of the well to the chip. Thus, a chip based amplifier may cleanly convert a pA signal to a mV signal. Common multichannel data acquisition boards can acquire this signal without difficulty.

In still another aspect, electronics are provided to measure voltage and/or current values for each of the wells. A controller may also be provided to control operation of the liquid dispensing system and the electronics. Further, a voltage source is coupled to the common electrode to create the voltage gradient.

In another embodiment, the invention provides a method for evaluating electrical currents flowing through ion channels of a plurality of cells. This method utilizes a plate having a plurality of wells that each have an end. At least some of the wells have an opening formed in the end, and a chamber is disposed below the plate and is filled with an electrolyte solution. A common electrode is also disposed in the chamber. With such a configuration, cells are dispensed in a solution into the wells. A pressure differential is applied between the wells and the chamber to collect cells into the openings and to create a high resistance seal between the cells and the ends of the wells. A potential difference is produced between the common electrode and well electrodes that are positioned within each well. Measurements of electrophysiological properties are taken from the cells that are positioned within the openings. Thus, a plurality of cells may be evaluated in parallel to create a high throughput screening system. Alternatively, cells may be deposited into the chamber and then drawn into the openings so that only a small portion of the cells are within the openings. The portions of the cells extending into the chamber may then be penetrated and measurements taken as previously described.

The invention further provides a method for evaluating electrical currents flowing through ion channels of the cell. The method utilizes at least one well having an end and a sidewall in the end that forms an opening through the end of the well. A glue-like substance is placed on the sidewall of the opening and one or more cells are deposited into the opening. The glue is used to create a high resistance seal between the cell and the sidewall opening formation. A potential difference is then created across the cell membrane and voltage and/or current measurements are taken and recorded. Hence, such a method produces a high resistance seal that is sufficient to make precise electrophysiological measurements. Additionally, such a technique permits the use of simple and inexpensive multi-well plates that are constructed of plastic or glass, rather than silicon and nitride or glass multi-usage plates.

One example of a procedure for performing a screening experiment is by providing a cell line with expressed target ion channels. Each well is configured to receive a few of these cells, although only one cell per well is necessarily measured. The plate is placed onto the chamber having an intracellular solution. The common electrode positioned in the chamber may be constructed of a metal plate that may be shifted to allow the solution to flow downward from each of the wells. Further, it will be appreciated that more than one common electrode may be used. For example, two or three common electrodes may be used. A slight pressure may be applied to each well, or a vacuum may be supplied to permit the cells to plug the through openings, thereby blocking them. Such procedure may take about 1 to 3 minutes to permit the cells to form high resistance seals with the holes in the end of each well.

The metal plate may then be shifted back to perforate the lower portion of the cells that are put through each well by the applied pressure. Alternatively, pressure pulses or a perforation solution may be used to perforate the cells. As another alternative, the cells may be penetrated by electroporation. After perforating the lower portion of the cells, the system is ready to record electrophysiological properties in a high throughput manner.

When the appropriate seal has been produced, a voltage of about −70 mV voltage difference is produced between the intracellular electrode (the common electrode that is formed from a metal plate) and each of the needles that are disposed in the well. The voltage is negative in this example because ground is defined to be at the exterior of the cell.

Before taking measurements, each well may be tested to determine whether the seal has been formed. If not, the well is labeled as a well having a “bad” seal (without a gigaseal) and may be discarded from subsequent considerations. The plate may be tested multiple times during the experiment to reconfirm the stability of seal formation. Applying small hyperpolarized pulses to the cell membranes may test each well. By excluding the wells without gigaseals from further consideration, ligands are effectively saved by applying them only to the “successful” wells.

Individual cell voltage and current measurements may then be taken and recorded using normal patch clamp electronics. The recorded data is stored and evaluated to determine the effectiveness of the compounds being tested. Further, the cells may be evaluated in a high throughput manner.

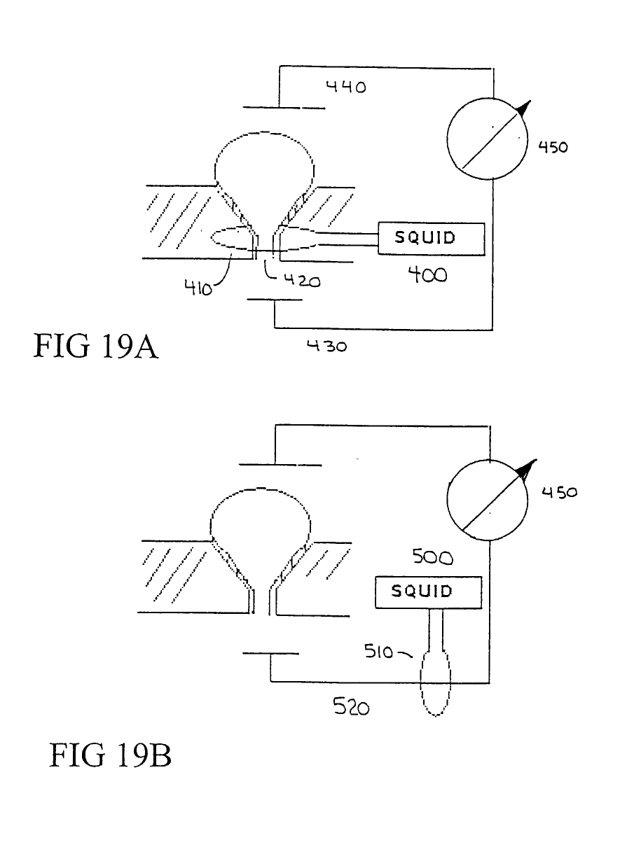

FIGS. 19A and 19B shows two methods of employing SQUID for the detection of ionic currents according to an embodiment of the present invention. A SQUID is a magnetic field sensor with high sensitivity. SQUID stands for “Superconducting QUantum Interference Device.” Using SQUID(s), small changes in magnetic fields can be measured very sensitively with high precision. Variants of SQUID are also suitable for the measurement of magnetic field gradients, voltages, current, and magnetic susceptibilities.

The output voltage of a SQUID is sinusoidal as a function of applied magnetic flux. Consequently, its behavior is nonlinear and periodic. One period corresponds with the magnetic field quantum Φ where Φ₀=2×10⁻¹⁵ Wb. With an input coil as current to flux transducer, a very sensitive current amplification can be obtained. Yet another way to increase SQUID sensitivity is to use a number of SQUIDS in series. When n such SQUIDS operate in phase, the output voltage increases in proportion to n, while the SQUID noise increases more slowly, in proportion to the square root of n.

One method of employing SQUID(s) according to an embodiment of the invention is to pass the current generated by an ion channel through a wire and thereby generate a magnetic field for the SQUID to detect as shown in FIG. 19B. Such a detector has the advantage of decoupling a current measurement from the voltage stimulation. A pneumatic electrical switch is used to switch the detector to different cells automatically without introducing excessive electrical noise. Thus, multiplexing, such as by using a recording device to sequentially record multiple wells, can be accomplished. Multiplexing is a term used to describe use of an input that can be switched to multiple sources to sample the measurements in sequence. Each recording device allows a reading from one well. Further, multiple recording devices can allow for parallel multiplexing.

Another method of employing SQUID(s) according to an embodiment of the invention is to measure the magnetic field when the channel is active. A SQUID sensor can sense the weak magnetic field that the ion channel current generates when it is placed near the cell. In one embodiment, an hourglass shaped capillary holds the cells or a SQUID sensor encircles an opening on a flat substrate or plate as shown in FIG. 19A.

In FIG. 19A, a SQUID device 400 is configured to detect changes in magnetic flux via loop 410 that encircles opening 420 and thus the path of ionic current. According to this embodiment, a SQUID device acts independently of the circuit comprising the two electrodes 430 and 440, and measuring device 450.

FIG. 19B shows a SQUID device 500 configured to detect changes in magnetic flux via loop 510, that encircles wire 520. According to this embodiment of the invention, a SQUID device detects a signal that may be physically displaced from the ion channel and or cell chamber.

The steps depicted in methods herein may be performed in a different order than as depicted and/or stated. The steps herein are merely exemplary of the order these steps may occur. The steps herein may occur in any order that is desired, such that the goals of the claimed invention are still achieved. Additionally, steps not desired to be used from the steps in the methods may be eliminated, such that the goals of the claimed invention are still achieved.

All patents and publications described herein are hereby incorporated by reference to the same extent as if each individual patent or publication was specifically and individually indicated to be incorporated by reference.

One skilled in the art would readily appreciate that the present invention is well adapted to carry out the objects and obtain the ends and technical advantages mentioned, as well as those inherent therein. The specific systems and methods described herein as presently representative of preferred embodiments are exemplary and are not intended as limitations on the scope of the invention. Changes therein and other uses will occur to those skilled in the art which are encompassed within the spirit of the invention are defined by the scope of the claims.

It will be readily apparent to one skilled in the art that modifications may be made to the invention disclosed herein without departing from the scope and spirit of the invention. The invention illustratively described herein suitably may be practiced in the absence of any element or elements, limitation or limitations that is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group. For example, if there are alternatives A, B, and C, all of the following possibilities are included: A separately, B separately, C separately, A and B, A and C, B and C, and A and B and C.

Thus, additional embodiments are within the scope of the invention and within the following claims.

What is claimed is:

1. A device for measuring electrophysiological properties of a cell membrane of an individual cell, said device comprising:
a plate provided with at least one opening, wherein said opening is bounded by a surface and wherein said surface is modified to facilitate formation of a gigaseal;
a chamber adjacent to said plate, wherein said chamber is in fluid communication with at least one opening and is adapted to hold a solution;
a first electrode;
a second electrode; and
wherein electrophysiological properties of a cell membrane of an individual cell is measured using said first electrode and said second electrode.

2. The device of claim 1, further comprising an amplifier in electrical contact with both electrodes.

3. The device according to claim 1, wherein said gigaseal is formed between a cell or cell membrane and said surface of said opening.

4. The device according to claim 1, wherein said plate comprises a well and a portion of said well is replaceable or interchangeable.

5. The device according to claim 4, wherein said replaceable portion comprises a disk having an opening.

6. The device according to claim 4, wherein sides of said well comprise plastic and a bottom of said well comprises glass.

7. The device according to claim 1, wherein said modification of said surface comprises chemically modifying said surface surrounding said at least one opening.

8. The device according to claim 7, wherein said chemical modification comprises covalently bonding a substance to the plate.

9. The device according to claim 1, wherein said substance is covalently bound to the well surface surrounding the opening.

10. The device according to claim 1, wherein said modification of said surface comprises modifying the surface surrounding said opening by heat treatment.

11. The device according to claim 10, wherein said plate comprises glass and said heat treatment comprises heating said surface to near or at a softening temperature of said glass.

12. The device according to claim 1, wherein said at least one opening is tapered.

13. The device according to claim 1, wherein said at least one opening comprises a counter bore and a through hole.

14. The device according to claim 1, wherein said cell is in a solution and said plate comprises a well, said device further comprising a multi-channel liquid dispensing system having a plurality of dispensers that are configured to place said solution in a well.

15. The device according to claim 1, further comprising a vacuum source coupled to said chamber to produce a vacuum within said chamber.

16. The device according to claim 1, further comprising electronics to measure voltage and/or current values for each of the wells.

17. The device according to claim 16, further comprising a SQUID detector.

18. The device according to claim 1 wherein said plate comprises a multi-well plate comprising an array of wells, wherein each of said wells comprises said opening.

19. The device according to claim 18, further comprising an automated liquid dispensing system, wherein each of said wells is independently addressable by said automated liquid dispensing system.

20. The device according to claim 1, wherein said electrophysiological properties of said cell membrane are recorded by measuring a current through said first and second electrode.

21. The device according to claim 1, wherein at least one of said electrodes comprises silver with silver chloride coating.

22. The device according to claim 1, wherein said solution is an electrically conductive solution.

23. The device according to claim 1, wherein said opening is created using a laser.

24. A device for measuring electrophysiological properties of a cell membrane of an individual cell, said device comprising:
a plate provided with at least one well, wherein said well is provided with an opening modified to receive an individual cell, wherein said opening is created using a laser and said opening is modified via heating;
a chamber adjacent to said plate, wherein said chamber is in fluid communication with said opening and is adapted to hold an electrically conductive solution;
a first electrode located in said chamber;
a second electrode located in said well; and
an amplifier in electrical contact with said first and second electrodes, wherein electrophysiological properties of a cell membrane of said individual cell are recorded by measuring a current through said first and second electrode.

25. The device according to claim 24, wherein said opening comprises a counter bore and a through hole.

26. The device according to claim 25, wherein said counter bore is drilled to a depth of approximately 80 to 110 μm.

27. The device according to claim 25, wherein said through hole has diameter of approximately 2 to 5 μm.

28. The device according to claim 24, further comprising a vacuum source coupled to said chamber to produce a vacuum within said chamber.

29. The device according to claim 24, further comprising a SQUID detector.

30. The device according to claim 24, wherein said plate comprises a multi-well plate comprising an array of wells, wherein each of said wells comprises an opening.

31. The device according to claim 30, further comprising an automated liquid dispensing system, wherein each of said wells is independently addressable by said automated liquid dispensing system.

32. The device according to claim 24, wherein said plate comprises a well and sides of said well comprise plastic and a bottom of said well comprises glass.

33. A removable disk comprising an opening wherein said disk serves as part of a well for use in measuring electrophysiological properties of a cellular membrane.

34. The disk according to claim 33, wherein said disk comprises glass.

35. The disk according to claim 33, wherein said disk comprises a plurality of openings.

36. The disk according to claim 33, wherein a surface surrounding said opening is chemically modified.

37. The disk according to claim 33, wherein a surface surrounding said opening is heat treated.

38. The disk according to claim 37, wherein said disk comprises glass and further wherein said heat treatment comprises heating said surface to near or at a softening temperature of said glass.

39. The disk according to claim 37, wherein said heat treatment comprises laser heating.

40. The disk according to claim 33, wherein said opening comprises a counter bore and a through hole.

41. The disk according to claim 40 wherein a size of said counter bore is approximately 130 μm and a size of said through hole is approximately 2 μm.

42. A method for evaluating currents flowing through ion channels of a cellular membrane, the method comprising:
providing at least one well comprising an opening having a modified surface to receive a cell comprising a cellular membrane;
depositing said cell onto said opening wherein said modified surface creates a gigaseal between said cell and said well; and
recording voltage and/or current measurements to evaluate said ion channel of said cell membrane.

43. The method according to claim 42, wherein sides of said well comprise plastic and a bottom of said well comprises glass.

44. The method according to claim 42, further using a vacuum source to produce a vacuum to assist in formation of said gigaseal.

45. The method according to claim 42, further comprising using an automated liquid dispensing system to deposit said cell, buffer and test compounds.

46. The method according to claim 42, wherein said modification of said surface comprises modifying the surface surrounding said opening by heat treatment.

47. The method according to claim 46, wherein said plate comprises glass and said heat treatment comprises heating said surface to near or at a softening temperature of said glass.

48. The method according to claim 42, wherein said modification of said surface comprises chemically modifying said surface surrounding said at least one opening.

49. The method according to claim 42, wherein said opening is created using a laser.

50. The method according to claim 42, wherein said at least one opening comprises a counter bore and a through hole.

51. The method according to claim 50, wherein said counter bore is created using said laser with a wavelength between approximately 150 and 300 nm.

52. The method according to claim 50, wherein said through hole is created using said laser with a wavelength between approximately 150 and 300 nm.

53. A method for creating a gigaseal, the method comprising:
providing at least one well comprising an opening;
depositing a solution comprising a plurality of cells into said well;
providing a positive pressure to said opening; and
providing a vacuum to said opening, creating a gigaseal between one of said plurality of cells and said opening.

54. The method according to claim 53, further comprising recording voltage and/or current measurements to evaluate an ion channel of a cell membrane of said one of said plurality of cells.

55. The method according to claim 53, wherein said opening is bounded by a surface and said surface is modified to assist in formation of said gigaseal.

56. The method according to claim 53, wherein said one of said plurality of cells comprises a good cell.

57. The method according to claim 53, wherein sides of said at least one well comprise plastic and a bottom of said at least one well comprises glass.

58. The method according to claim 53, wherein said surface is modified by heat treatment.

59. The method according to claim 58, wherein a plate said at least one well and said plate comprises glass and said heat treatment comprises heating said surface to near or at a softening temperature of said glass.

60. The method according to claim 53, wherein said opening comprises a counter bore and a through hole.

61. The method according to claim 53, wherein said opening is created using a laser.
